(19) United States
(12) Patent Application Publication
(10) Pub. No.: US 2014/0275614 A1
(43) Pub. Date: Sep. 18, 2014

(54) PRODUCTION AND PURIFICATION OF CARBOXYLIC BETAINE ZWITTERIONIC MONOMERS

(71) Applicant: Zwitter Technology, LLC, Seattle, WA (US)

(72) Inventors: Yuting Li, Seattle, WA (US); Hong Xue, Pleasanton, CA (US); Yingquan Song, Sammamish, WA (US)

(73) Assignee: Zwitter Technology, LLC, Seattle, WA (US)

(21) Appl. No.: 14/212,407

(22) Filed: Mar. 14, 2014

Related U.S. Application Data
(60) Provisional application No. 61/783,465, filed on Mar. 14, 2013.

(51) Int. Cl. C07C 227/40

(52) U.S. Cl. CPC C07C 227/40
USPC 560/218

(57) ABSTRACT

A method is provided for the production and purification of carboxylic betaine-based zwitterionic vinyl monomers, such as (meth)acrylic or (meth)acrylamide monomer containing a betaine-type group of the formula:

[chemical structure]

wherein A is an ester, amide, benzyl, pyridinyl, or imidazolyl; R is hydrogen or methyl; and n is an integer from 0 to 10.

The method involves the reaction of acrylic acid with aminoalkyl (meth)acrylate or N-amino-alkyl (meth)acrylamide having a basic tertiary nitrogen atom in the presence of a free radical inhibitor, this reaction leads to a betaine:acid complex that can be either separated from the reaction media into pure betaine:acid complex powder or further purified into pure monomer powder by passing the reaction mixture through a base resin followed by precipitation in a nonsolvent or direct precipitation in a nonsolvent containing an organic base.

### REFERENCE TO RELATED APPLICATIONS

This application is a Non-Provisional of U.S. Provisional Patent Application Ser. No. 61/783,465, filed Mar. 14, 2013. The entire content of the above application is hereby incorporated by reference.

### GOVERNMENT LICENSE RIGHTS

This invention was made with Government support under Contract Numbers IIP-1013431 and IIP-1127475 awarded by the National Science Foundation. The Government has certain rights in the invention.

### FIELD OF THE INVENTION

This invention involves the production and purification of carboxylic betaine-based zwitterionic monomers.

### BACKGROUND OF THE INVENTION

The betaine compound having both a cationic group and an anionic group within its molecule has been attracting attention because of this structural feature. Conventional methods for producing betaine-type compounds are by reaction of a tertiary amine with beta-propiolactone (see, for example, Fiedorek, 1960, U.S. Pat. No. 2,548,428) and by reaction of a tertiary amine with an alkali-metal salt of a halogenated monocarboxylic acid (see, for example, Schuller et al, U.S. Pat. No. 2,958,682). For the first case, the use of toxic and expensive reagent such as beta-propiolactone makes it difficult for large-scale production. For the latter case, strict thermal and pH reaction conditions are required, making it hard to achieve higher polymer conversion and yield.

Japanese Kokai Publication Hei-5-32600 discloses a process for producing a betaine compound which comprises reacting an aliphatic primary amine with a quaternary ammonium compound having a defined structure at 6≦pH<8 and further reacting the reaction product with a defined halogenated lower carboxylic acid at 6≦pH<8. The betaine compound produced by this process has foaming and detergent properties so that it can be used as a surfactant for hair and body cleansing.

Japanese Kokai Publication Hei-5-294905 discloses a carbobetaine compound and a process for its production. This process for producing a carbobetaine compound comprises reacting a defined amino compound with a defined halogen-containing salt. The carbobetaine compound obtained by this process has an emolient action on hair and skin for hair care and skin care products.

U.S. Pat. No. 4,012,437 (1977) and U.S. Pat. No. 3,689,470 (1972) describe the synthesis of the carboxylic betaine methacrylate (CBMA) by reacting dimethylaminoethyl methacrylate (DMAEMA) with acrylic acid in aqueous media or in organic solvent. However, the reaction media was used directly for further polymerization, no monomer purification process was described.

### SUMMARY OF THE INVENTION

The present invention, developed in light of the above state of the art, is to provide a purification method which can provide pure zwitterionic monomers, having the following structure in a solid powder state.

[chemical structure]

### BRIEF DESCRIPTION OF THE DRAWING

FIG. 1 is an ¹H NMR spectrum of the carboxybetaine methacrylate (CBMA) monomer.

FIG. 2 is a ¹H NMR spectrum of the carboxybetaine acrylamide (CBAA) monomer.

FIG. 3 is a ¹H NMR spectrum of the carboxybetaine methacrylamide (CBMAA) monomer.

FIG. 4 is a representative surface plasmon resonance (SPR) sensorgrams showing ultra low non-specific protein adsorption to gold surfaces coated with polyCBMA (monomer produced according to procedure in example 4) via atom-transfer radical polymerization (ATRP) upon exposure to either fibrinogen or lysozyme, i.e., 0.2 ng/cm² for fibrinogen and 0.1 ng/cm² for lysozyme. Ultra low fouling is defined as <5 ng/cm² adsorbed proteins.

FIG. 5 is a representative SPR sensorgrams showing ultra-low non-specific protein adsorption to gold surfaces coated with polyCBAA (monomer produced according to procedure in example 6) via ATRP upon exposure to either undiluted blood plasma or serum, i.e., 3.6 ng/cm² for plasma and 2.2 ng/cm² for serum.

FIG. 6 is a continuous synthesis flow chart.

### DETAILED DESCRIPTION ON THE INVENTION

According to particular aspects, methods to produce and purify the carboxylic betaine monomer into a dry powder are herein disclosed. According to further particular aspects, the invention disclosed herein relates to purification methods that can separate carboxylic betaine monomer from its reaction mixture and a dry solid powder can be obtained.

The carboxylic betaine based zwitterionic monomer can be synthesized via the reaction of acrylic acid and the corresponding tertiary amine based monomers.

[chemical structure]

The reaction of a tertiary amine and acrylic acid can be carried out with or without solvent. The total concentration of acrylic acid and amine in the solvent medium can be anywhere from 5 to 99% by weight and is preferably about 60 to 95% by weight. Generally, a relatively large ratio of acid to amine is preferred in order to favor the shift of the equilibrium toward the formation of the betaine. For practical purposes the ratio of acid to amine is from 0.1:1 to 10:1 and preferably 1:1 to 5:1. For example, 2:1 to 3:1 mole ratios can provide equilibrium conversions of amine to betaine as high as 80-90%.

For the production of polymerizable monomeric betaines, the use of two moles of acrylic acid for each mole of the tertiary amine such as dimethylaminoethyl methacrylate often produces what appears to be a complex of one mole of the betaine with one mole of the acid. This betaine:acid complex can be separated from the reaction media in the presence of organic solvent (e.g., hexane, acetone, or THF) with or without adding additional acids (e.g., HCl or H₂SO₄) to the reaction mixture into a white crystal solid. This betaine:acid complex (where acid can be HCl, H₂SO₄ or acrylic acid) can be used directly for further applications.

Further purification can be applied to obtain pure betaine monomer. This purification process can be carried out using the reaction mixture directly or the separated betaine:acid as the starting materials. The acid can be removed from the reaction media or the betaine:acid complex by using an inorganic base, organic base or base ion exchange resin. The base resin can be any type as far as they can remove the acid from the zwitterionic monomer/acid complex. The reaction mixture can be diluted with anhydrous solvent such as methanol or ethanol before passing through the base resin. The monomer concentration in the diluted reaction mixture can be in the range of 10-90%. The solutions after ion exchange resin treatment can be concentrated under vacuum, and then added into nonsolvent such as anhydrous THF, dioxane, chloroform, dichloromethane and acetone for the precipitation of the monomer. A white powder can be obtained by this separation method.

According to certain aspects, for the separation of the zwitterionic monomer from its complex with the acid is to use organic base, such as trialkylamine. Anhydrous organic solvent such as THF, dioxane, chloroform, dichloromethane or acetone can be mixed with the organic base, and then added to the reaction mixture. Zwitterionic monomer can be precipitated out from the mixture.

These zwitterionic monomers can also be manufactured via a continuous process. The tertiary amine based monomer and the acrylic acid are pumped into a reactor from their corresponding stock solutions. The reaction mixture is then pumped into a precipitation flask, where a precipitation solvent (e.g., mixture of organic solvent and trialkylamine base) is also pumped in at the same time. White solid can be formed quickly in the precipitation flask. These solid/liquid mixtures can be transferred into a filtration device to separate the solid from the liquid. All the above process can be set to run continuously.

The obtained monomer purity can be assessed by ¹H NMR. FIGS. 1-3 show the NMR spectra of the obtained CBMA, CBAA, and CBMAA monomers. The quality of these monomers can be further assessed using protein adsorption monitored by surface plasmon resonance (SPR) sensors when they are polymerized onto SPR surfaces. Nonspecific protein adsorption on these zwitterionic polymer coated gold surfaces are lower than 5 ng/cm² for single-protein solution and undiluted blood plasma and serum.

The following modes of operation are suggested by way of illustration.

### EXAMPLES

### Example 1

DMAEMA (1.0 mol, 170 mL) was added to a 2.0 L flask equipped with a mechanical stirrer, the flask was then cooled to 0° C. using an ice bath. Acrylic acid (2.0 mol, 140 mL) was added dropwise to the flask within 30 minutes. The reaction was carried out at 0° C. for another 30 minutes, then at room temperature for 4 hours. The reaction solution became gradually more viscous indicating the reaction was ongoing. The above solution was then diluted with 100 mL ethanol and stirred overnight. Hexane (500 mL) was then added under stirring into the reaction mixture. The flask containing the above mixture was sealed and left at room temperature. Within a few days, a lot of white solid precipitated from the mixture. The solid was then filtered and washed with hexane and dried under vacuum. NMR analysis showed that it was a betaine:acid complex.

### Example 2

DMAEMA (1.0 mol, 170 mL) was added to a 2.0 L flask equipped with a mechanical stirrer, the flask was then cooled to 0° C. using an ice bath. Acrylic acid (2.0 mol, 140 mL) was added dropwise to the flask within 30 minutes. The reaction was carried out at 0° C. for another 30 minutes, then at room temperature for 4 hours. The reaction solution became gradually more viscous indicating the reaction was ongoing. The above solution was then diluted with 100 mL ethanol and stirred overnight.

Amberlyst@21 (650 grams; 4.6 mmol/g; 3.0 eq) (a weak ion exchange resin) suspended in 1.0 L ethanol was then added to the above monomer solution under stirring at room temperature to neutralize the excess amount of acrylic acid. After 3 hours, the resin was filtered off and washed with 100 mL ethanol. The solvent was removed using a rotary evaporator. The obtained viscous solution was then slowly added to dry acetone (10 times the volume of solution) under stirring. White CBMA crystals precipitated from the mixture. The solid was then filtered, washed with dry ether, and dried under vacuum. The total yield was about 70% (150 grams).

### Example 3

DMAEMA (1.0 mol, 170 mL) together with 0.2 g radical inhibitor was added to a 2.0 L flask (FIG. 1) equipped with a mechanical stirrer, the flask was then cooled to 0° C. using an ice bath. Acrylic acid (2.0 mol, 140 mL) was added dropwise to the flask within 30 minutes. The reaction was carried out at 0° C. for another 30 minutes, then at room temperature for 4 hours. The reaction solution became gradually more viscous indicating the reaction was ongoing. The above solution was then diluted with 100 mL ethanol and stirred overnight. The above solution was then diluted with another 200 mL methanol, 600 mL acetone, and 200 mL triethylamine was then added under stirring. After 30 minutes, a white solid precipitated from the mixture. The solid was then filtered, washed with dry acetone, and dried under vacuum. The total yield was about 75%.

### Example 4

Dimethylaminoethyl methacrylate (DMAEMA, 0.48 mol, 76 g), together with 0.14 g radical inhibitor were added to a 2.0 L flask (FIG. 1) equipped with a mechanical stirrer, the flask was then cooled to 0° C. using an ice bath. Acetic acid (0.47 mol, 27 mL) and acrylic acid (0.95 mol, 65 mL) were sequentially added dropwise to the flask within 30 minutes. The reaction was carried out at 0° C. for another 30 minutes, then at room temperature for 4 hours. The reaction solution became gradually more viscous indicating the reaction was ongoing. Acetone (50 mL) was added to dilute the reaction mixture and the reaction mixture was stirred overnight. To the above solution was added over 1 hour a 1:1 volume ratio of acetone/triethylamine solution under stirring. A white solid precipitated from the mixture. The solid was then filtered out, washed with dry acetone, and dried under vacuum. The total yield was about 75%.

### Example 5

Dimethylamino propyl acrylamide (DMAPA, 1.0 mol, 156 g) together with 0.2 g radical inhibitor was added to a 2.0 L flask (FIG. 1) equipped with a mechanical stirrer, the flask was then cooled to 0° C. using an ice bath. Acrylic acid (2.0 mol, 140 mL) was added dropwise to the flask within 30 minutes. The reaction was carried out at 0° C. for another 30 minutes, then at room temperature for 4 hours. The reaction solution became gradually more viscous indicating the reaction was ongoing. The above solution was then diluted with 100 mL ethanol and stirred overnight. The above solution was then diluted with another 200 mL methanol, 600 mL acetone, and 200 mL triethylamine was then added under stirring. After 30 minutes, a white solid precipitated from the mixture. The solid was then filtered, washed with dry acetone, and dried under vacuum. The total yield was about 78%.

### Example 6

Dimethylamino propyl acrylamide (DMAPA, 1.0 mol, 156 g) and 200 mL methanol, together with 0.2 g radical inhibitor were added to a 2.0 L flask (FIG. 1) equipped with a mechanical stirrer, and the flask was then cooled to 0° C. using an ice bath. Acetic acid (1.0 mol, 57 mL) and acrylic acid (1.0 mol, 70 mL) were sequentially added dropwise to the flask within 40 minutes. The reaction was carried out at 0° C. for another 30 minutes, then at room temperature for overnight. To the above solution was added over one hour a 1:1 volume ratio of acetone/triethylamine solution under stirring. A white solid precipitated from the solution. The solid was then filtered, washed with dry acetone, and dried under vacuum. The total yield was about 75%.

### Example 7

Dimethylamino propyl methacrylamide (DMAPMA, 1.0 mol, 168 g) together with 0.2 g radical inhibitor was added to a 2.0 L flask (FIG. 1) equipped with a mechanical stirrer, the flask was then cooled to 0° C. using an ice bath. Acrylic acid (2.0 mol, 140 mL) was added dropwise to the flask within 30 minutes. The reaction was carried out at 0° C. for another 30 minutes, then at room temperature for 4 hours. The reaction solution became gradually more viscous indicating the reaction was ongoing. The above solution was then diluted with 100 mL ethanol and stirred overnight. The above solution was then diluted with another 200 mL methanol, 600 mL acetone, and 200 mL triethylamine was then added under stirring. After 30 minutes, a white solid precipitated from the mixture. The solid was then filtered, washed with dry acetone, and dried under vacuum. The total yield was about 70%.

### Example 8

### Continuous Synthesis

Stock solution A: 3.9 M DMAPA, 7.8 mM hydroquinone, in MeOH; Stock solution B: 3.9 M acetic acid, 3.9 M acrylic acid, in MeOH; Stock solution C: 1:1 volume TEA/acetone. DMAPA (1 mol) was added to a 2 liter flask, followed by 200 mL methanol and 0.23 g hydroquinone. The mixture was cooled with an ice bath. Acetic acid (1 mol) and 1 mol acrylic acid were slowly added to the flask and was stirred at room temperature for 24 hours.

Stock solution A and B were then pumped into the reaction flask at a speed of 0.15 mL/minute. At the same time, the reaction mixture was pumped out at a speed of 0.30 mL/minute to a precipitation flask (containing 60 mL stock solution C), stock solution C was added into the precipitation flask at a speed of 0.9 mL/minute via another pump.

When the reaction mixture and precipitating agent are pumped into the flask, it gets full and the mixture overflows to the filtration funnel. CBAA monomer solid was collected in the filtration funnel.

For all the examples (Examples 1-8) shown above, the monomer purity could be increased by a further purification cycle such as dissolving the monomer into methanol, and precipitation in an inorganic solvent in the presence of organic base.

### Example 9

### Protein Adsorption Measurements

Glass chips were first coated with an adhesion-promoting chromium layer (thickness 2 nm) and a surface plasmon active gold layer (48 nm) by electron beam evaporation under vacuum. Before self-assembling monolayer (SAM) preparation, the substrates were washed with pure ethanol, cleaned under UV light, and washed with water and pure ethanol. SAMs were formed by soaking gold-coated substrates in pure ethanol solution of ω-mercaptoundecyl bromoisobutyrate at room temperature after careful cleaning.

The substrate with immobilized initiators was then placed in a reaction tube, sealed with rubber septum stoppers, and degassed with nitrogen for 30 minutes. Degassed CBMA solution (pure water and methanol in a 1:1 volume ratio) and bipyridine (BPY)/CuBr solution were then transferred to the tube using syringe under nitrogen protection. After the reaction, the substrate was removed and rinsed with ethanol and water, and the samples were kept in water overnight. Rinsing with phosphate buffered saline (PBS) buffer was also applied to remove unbound polymers before testing.

Protein adsorption was measured with a custom-built SPR sensor based on wavelength interrogation. A SPR chip was attached to the base of the prism, and optical contact was established using refractive index matching fluid (Cargille). A dual-channel flow cell with two independent parallel flow channels was used to contain the liquid sample during experiments. A peristaltic pump (Ismatec) was utilized to deliver the liquid sample to the two channels of the flow cell. A fibrinogen solution of 1.0 mg/mL in PBS (0.15 M, pH 7.4) was flowed over the surfaces at a flow rate of 0.05 mL/minute. Protein adsorption from lysozome, undiluted blood serum or plasma was also performed in a similar way. A surface-sensitive SPR detector was used to monitor protein-surface interactions in real time. In this study, wavelength shift was used to measure the change in surface concentration (mass per unit area).

What is claimed is:

1. A purification method for the production of zwitterionic monomers having carboxylic betaine structures.

2. The zwitterionic moiety of the above compounds features an N atom connected through two methylene groups to a carboxylic group. They are made from the conjugate addition of N,N-diaminoalkyl (meth)acrylate or (meth)acrylamide to acrylic acid.

3. The purification method includes passing the reaction mixture through an ion exchange base resin followed precipitation in a nonsolvent.

4. The purification can also be achieved by precipitating the reaction mixture into a nonsolvent containing an organic base to remove the remaining acid.

5. The nonsolvent in claims 3 and 4 can be tetrahydrofuran, chloroform, dioxane, acetone, dichloromethane, toluene as well as DMF.

6. The organic bases used in claim 4 are amines having the structure NR1R2R3, where R1, R2 and R3=H, Cl˜12.

7. The purity of zwitterionic vinyl monomers can be evaluated by 1H NMR. Typical monomer purity is >93%. Purity >99% can be achieved by further purification. The quality of the monomer can be further monitored via protein adsorption from single protein solutions or undiluted blood plasma or serum. The typical protein adsorption is <5 ng/cm2 and as low as 0.3 ng/cm2 can be achieved.

8. The zwitterionic monomer can be produced via a continuous process.
